(19) United States
(12) Patent Application Publication
(10) Pub. No.: US 2025/0041018 A1
(43) Pub. Date: Feb. 6, 2025

(54) MULTIPLE NEEDLE INJECTOR

(71) Applicant: Allergan Sales, LLC, North Chicago, IL (US)

(72) Inventor: Jacob G. Unger, Nashville, TN (US)

(21) Appl. No.: 18/741,546

(22) Filed: Jun. 12, 2024

Related U.S. Application Data
(63) Continuation of application No. 16/594,692, filed on Oct. 7, 2019, now abandoned.
Application No. 16/594,692 is a continuation of application No. 15/066,949, filed on Mar. 10, 2016, now Pat. No. 10,433,928.
(60) Provisional application No. 62/131,064, filed on Mar. 10, 2015.

(51) Int. Cl. A61B 90/00; A61B 17/00; A61F 2/12; A61M 5/32

(52) U.S. Cl. CPC A61B 90/02; A61B 2017/00796; A61F 2/12; A61F 2250/0003; A61M 5/3298; A61M 2206/20

(57) ABSTRACT

A fluid injection device can include a plurality of needles, a hub from which the needles project, and a base component. The hub can have a directing portion extending therefrom. The plurality of needles can each be in fluid communication with a respective needle base, and the needle bases can at least partially surround the directing portion. The directing portion can extend in a direction away from the hub for diverting flow toward the plurality of needle bases. The base component can be separate from and coupleable to the hub such that when coupled thereto, the hub directing portion extends into a cavity of the base component upstream of the plurality of needle bases.

### CROSS-REFERENCES TO RELATED APPLICATIONS

This application is a continuation of U.S. patent application Ser. No. 16/594,692, filed Oct. 7, 2019, which is a continuation of U.S. patent application Ser. No. 15/066,949, filed Mar. 10, 2016 (now U.S. Pat. No. 10,433,928), which claims the benefit of 62/131,064, filed Mar. 10, 2015, each of which is incorporated herein by reference in its entirety.

### FIELD OF THE INVENTION

The present invention is generally directed to an apparatus using a plurality of needles to increase flow rates during tissue expansion and injection of fluids into subcutaneous tissue and the method thereof.

### BACKGROUND OF THE INVENTION

Breast reconstruction Is one of the most common procedures performed in plastic surgery today. One out of eight women will have breast cancer in their lifetime, the majority of whom will undergo reconstruction. Currently federal law mandates that any woman who has breast cancer must be offered reconstruction. Last year 96,000 breast reconstructions were performed, the vast majority of which were expander implant based reconstructions. Of note, many cases are bilateral, and therefore over 100,000 expanders are being utilized in breast reconstruction ever year.

The typical breast reconstruction process involves placing a deflated tissue expander in the chest pocket after mastectomy (breast removal). The expander includes a port through which sterile saline can be forced, causing the volume of the expander to increase. The expander is usually filled with sterile saline in the operating room prior to closing the surgical opening in the skin. The patient then returns to the clinic two weeks later for further expansion. Because the port is covered by skin, the port is located with the aid of a port locator magnet. Once it is located, a needle is placed through the skin into the port and sterile saline is injected. This procedure is done on a weekly basis until the skin envelope is expanded lo a large enough size to accommodate the desired implant size for an appropriately sized breast mound. Typically, the expansion procedure is performed four to eight separate times prior to being ready for the expander/implant exchange.

This procedure is not limited to breast reconstruction. Tissue expanders are also used in bum reconstruction and various other types of reconstruction where skin expansion is needed.

The most pressing issue associated with breast and other types of tissue expansion is the exceedingly min-ow needle used in current systems, such as the MENTOR brand winged infusion set, as compared to the pipe diameter of the rest of the system. The entire system is bottlenecked by the 21 gauge needle that is used to inject the port. The inner diameter of the needle is 0.51 mm (outer diameter 0.81 mm), which is the maximum allowable gauge needle per the manufacturer instructions (see Mentors Product Website) due to the nature of the port. Narrow needles result in slow flow of sterile saline, and require more time to fill the tissue expander. The filling process in the operating room can be as long as 10 to 15 minutes during which time the surgeons and nurse must patiently wait for the expander to fill with the patient still open. Simply a larger needle would core out the silicone and make the port leak, and this fail.

### SUMMARY OF THE INVENTION

We disclose herein a fluid injector apparatus for injecting fluid through a plurality of needles into a subcutaneous port during tissue expansion comprising a base, tubing connecting the needles, and a base fluid injection system to increase linear flow velocity and decrease procedure time.

We also disclose a method of injecting fluid through a plurality of needles into a subcutaneous port during tissue expansion comprising a base, tubing connecting the needles, and a base fluid injection system to increase linear flow velocity and decrease procedure time.

### BRIEF DESCRIPTION OF THE DRAWINGS

Further advantages of the invention will become apparent by reference to the detailed description of preferred embodiments when considered in conjunction with the drawings.

FIG. 1 is a top plan view of the multiple injector needle apparatus.

FIG. 2 is a front view of the multiple injector needle apparatus.

FIG. 3 is a front view of a plastic sleeve for needle protection.

FIG. 4 is a perspective view of the multiple injector needle apparatus.

FIG. 5 is a top plan view of the multiple injector needle apparatus.

FIG. 6A-6D are views of an embodiment of the multiple injector needle apparatus. Independently, FIG. 6A is a perspective view of the hub of the apparatus. FIG. 6B is a cross-sectional view of the multiple injector needle apparatus. FIG. 6C is a top plan view of the hub of the apparatus. FIG. 6D is a cross sectional view of the hub of the apparatus taken along the line 6D-6D of FIG. 6B.

FIG. 7A-7D are views of an embodiment of the multiple injector needle apparatus. Independently, FIG. 7A is a perspective view of the hub of the apparatus. FIG. 7B is a top plan view of the hub of the apparatus. FIG. 7C is a cross-sectional view of the aggregating cone of the apparatus taken along the line 7C-7C of FIG. 7B. FIG. 7D is a cross-sectional view of the aggregating cone of FIG. 7C of the apparatus at increased magnification.

FIG. 8A-8C are views of an embodiment of the apparatus with needles inserted into the hub. Independently, FIG. 8A is a perspective view of the hub of the apparatus. FIG. 8B is a right side view of the hub of the apparatus. FIG. 8C is a cross-sectional view of the needles and hub of the apparatus.

FIG. 9A-9C are views of an embodiment of the apparatus with needles inserted into the hub. Independently, FIG. 9A is a perspective view of the hub of the apparatus. FIG. 9B is a right side view of the hub of the apparatus. FIG. 9C is a cross-sectional view of the aggregating cone of the apparatus.

FIG. 10 is a cross sectional view of an embodiment of the hub of the apparatus.

FIG. 11 is a cross sectional view of an embodiment of the hub of the apparatus with needles inserted.

### BRIEF DESCRIPTION OF THE DRAWINGS

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

To overcome the issue of the slow flow through a single injection needle, our invention uses a plurality of needles. The typical injection port is nearly 3 cm in diameter. So, while a large single needle would cause failure, multiple small needles can easily be placed into the silicone dome at the same time without breaking the seal. The present invention (as pictured in FIG. 2) would solve the exceedingly small needle issue by allowing for a four (4) times increase in the flow velocity as compared to the current standard and sole existing option by allowing a plurality of separate needles contained within one construct at the end of butterfly tubing thus resulting in immense time savings in the operating room as well as the clinic.

We disclose herein such an apparatus that allows for increased flow rate of fluids into a tissue expander. The apparatus has multiple needles that can be simultaneously inserted into the port, thus allowing more fluid to flow into the expander. As a result of the increased flow, the time to fill the tissue expander is significantly reduced.

Referring to the drawings, FIG. 2 illustrates the multiple needle apparatus 1 consisting primarily of needles 2 projecting outward from a hub 5 and tubing 4 for connecting the hub to a fluid source.

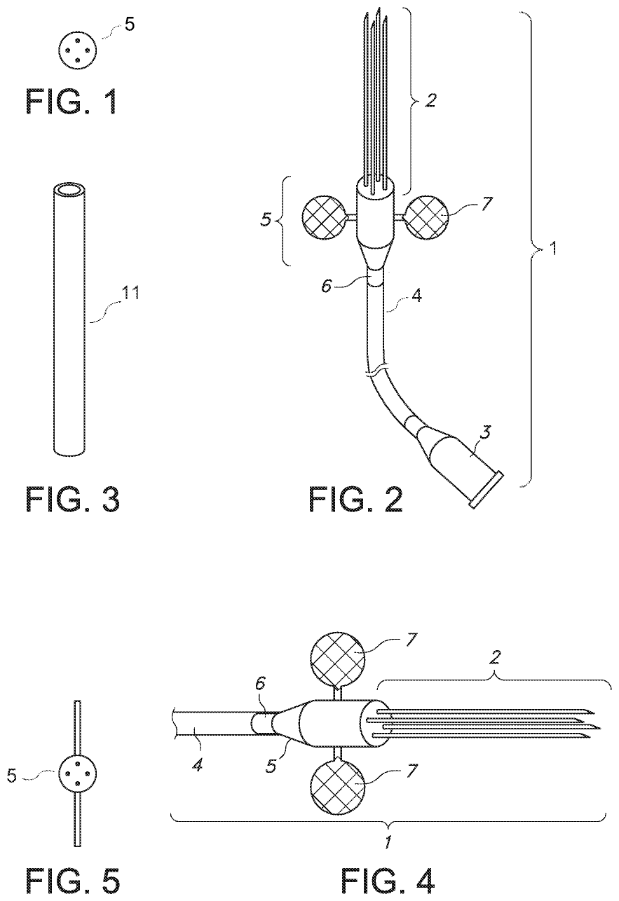

The needles 2 project from the first end (injection side) of the hub 5 such that they are substantially parallel to each other. As shown in FIG. 1 and FIG. 4, the needles are spaced such that all of them can simultaneously enter the injection port on the breast tissue expander. In the example shown, the needles are spaced no more than five (5) millimeters across. The needles 2 may be shorter than currently used needles, or they may have ultra-thin walls, thus reducing the pressure required for increased flow of fluid into the tissue expander. Any type and size of needle that allows the flow of liquid may be used in the device, although in a preferred embodiment, a standard hypodermic will be used in the apparatus 1.

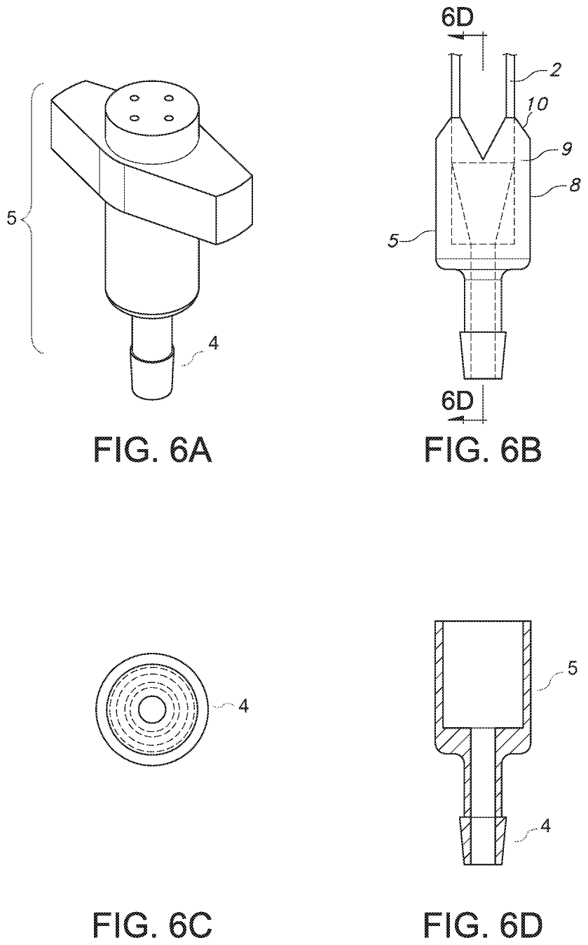

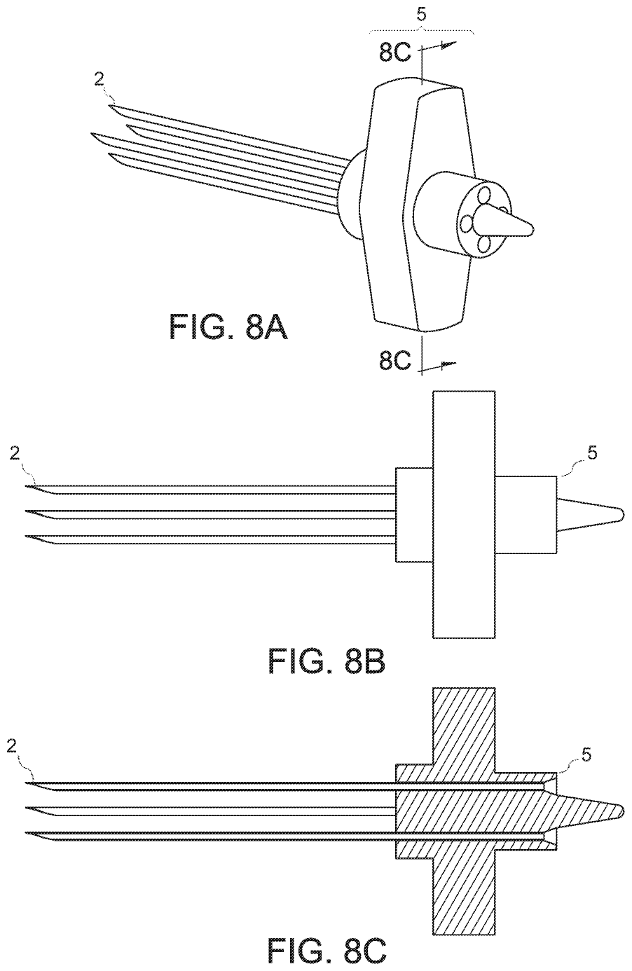

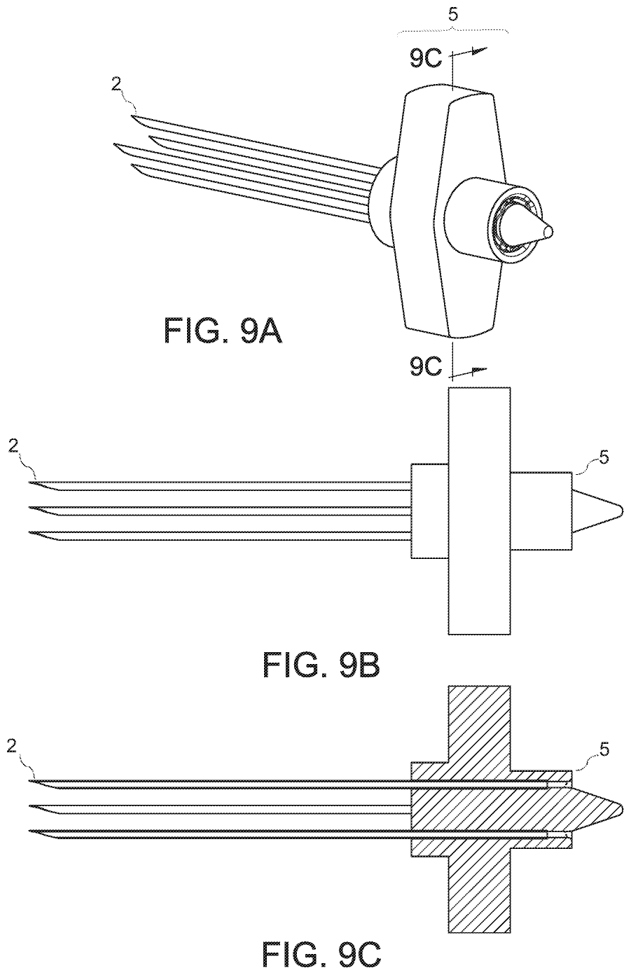

It should be appreciated that any size hub 5 may be selected to accommodate a plurality of needles. In the embodiments depicted in FIG. 6, the hub 5 may be as large as three (3) centimeters in diameter or as small as one-quarter (0.25) of a centimeter in diameter. Also, the needles may be secured into the hub by pressure as shown in FIG. 8A-8C or secured into the hub by glue as shown in FIG. 9A-9C.

Opposite the needle side of the hub 5 lies the second end 6 of the hub. The second end 6 is configured to receive IV tubing. In the example shown, butterfly IV tubing would connect to the second end 6. As shown in FIG. 6A, the hub 5 ideally would be made of plastic, and would have plastic handles 7 that would aid in the insertion of the needles into the injection port.

As shown in FIG. 4, the preferred embodiment of the apparatus has four 4.5 cm long 21 gauge needles 2 situated parallel allowing precise placement into the silicone dome of the subcutaneous port during a tissue expansion procedure, thus increasing linear flow rate into the apparatus 1. In this embodiment, the actual diameter of the preferred embodiment of the hub 5 is approximately 5 millimeters. Tubing 4 attaches to hub 5 at second end 6 by stretching the tubing over the second end 6 of the hub. It should be appreciated that although the hub is shown as having a permanent connection to the tubing, it can be constructed such that the tubing is detachable. Tubing 4 terminates at the other end in a female connection of the leur lock base 3.

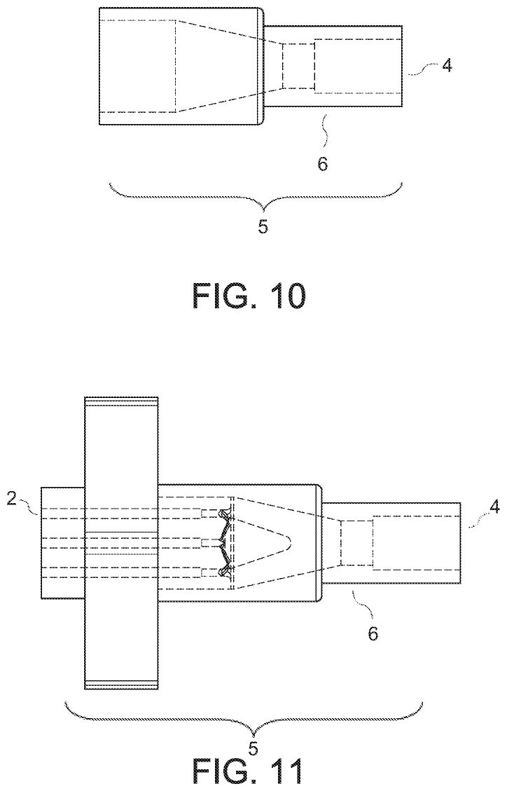

As shown in FIG. 10, a potential embodiment of the tubing 4 connection to the hub 5 includes the entry portal for the tubing 4 having an inner diameter equal to the outer diameter of the tubing 4. The tubing 4 is securely attached to the second end 6 of the hub 5 allowing unimpeded fluid flow through the tubing 4 into the hub 5, as shown in FIG. 11.

To use the apparatus 1, leur lock base 3 is attached to a fluid source, such as a syringe. The fluid is then pushed through the tubing 4.

As shown in FIG. 6A-6D, the preferred embodiment of the apparatus 1 has a graded directing wall 8 with the narrowest portion at the fluid entry point (diameter of the tubing 4) and the widest portion at the fluid exit point (the plurality of needles 2) as pictured in FIG. 6B. As shown FIG. 6C, the gradual change in diameter of the system created by this angled directing wall 8 improves laminar flow.

As the fluid passes through the hub 5, it is distributed into the plurality of needles 2 via a directing cone 9. The directing cone 9 encourages and directs flow from the proximal end of the hub 5 into the needles 2. Without the directing cone 9, the fluid would require a 90 degree change in direction to enter the plurality of needles 2. Therefore, the increased direction.al flow in. a graduated manner created by the directing cone 9 improves laminar flow, thus improving the actualized flow rate of the apparatus 1.

When the needles are filled with fluid, the needles are inserted into the injection port of the tissue expander, and the fluid then fills the tissue expander.

Potential embodiments of the needles 2 including a plurality of hypodermic needles, conical needles, taper needles or 1.5 inch needles where the diameter actually tapers from a larger gauge (i.e. 18 gauge) to a smaller 21 gauge size by point in the needle where the port puncture occurs, as the 21 gauge segment is required only for the portion that enters the port. Additionally, thin walled needles approaching six (6) times thinner than the standard needle wall are contemplated as an embodiment of the instant invention.

Another potential embodiment of the needles 2 includes ultra-thin wall 21 gauge needles that maintain the same external diameter of 0.81 millimeter, but a larger internal diameter.

As shown in FIG. 2, the preferred embodiment of the invention has a leur lock base 3 to allow for secure attachment of a syringe or other fluid source.

As shown in FIG. 2, the preferred embodiment of the invention consists of approximately 11 inches of IV tubing 4 to connect the needles. It should be appreciated, however, that the apparatus 1 can include any type or length of tubing 5 capable of connecting the needles 2.

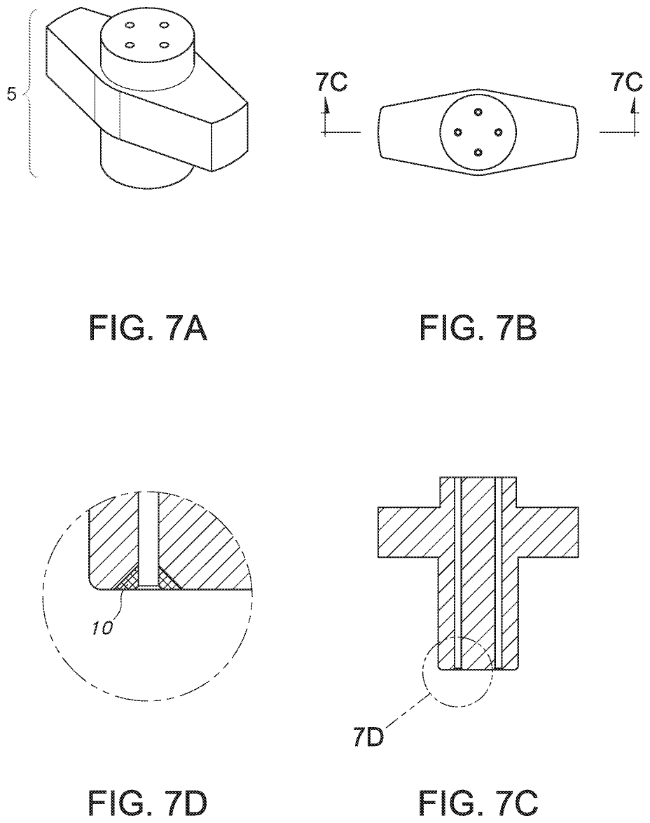

As shown in FIG. 6B and FIG. 7C-7D, a potential embodiment of the invention consist of a cone-shaped cut out in the hub 5 that directs fluid flow from the base 3 into the needle 2 in a graduated manner. As shown in FIG. 7D, this aggregating cone 10 has a diameter equal to one half of the internal diameter of the hub 5 minus the diameter of the directing cone 9 (Formulaically: D_{AC}=½ (D_{hub}−D_{DC}). Thus, surrounding each needle base, there is a graded wall, consisting of an internal gradual decrease in the diameter of the base leading to the needle, to channel fluid flow from the widest portion of the directing wall 8 to the base of the directing cone 9 and increase laminar flow rates.

Other potential embodiments of the IV tubing 4 include no rolling ball lock to stop IV flow; wider tubing to allow improved flow especially in the butterfly segment; a series of one-way valves instead of the four way stopcock to create a “no-touch” system allowing drawing and injecting into the port without manually turning the stocking into the correct position; and an autoinjector that the syringe with a preset amount of saline (i.e. 35, 50, or 60 cc). The one-way valves would require two one-way valves allowing for inflow from the IV bag and out flow through the needle side, and the autoinjector could be created with a plastic handle and preloaded spring that draws back on the syringe to the desired level.

In another potential embodiment, the invention can be used in subcutaneous ports requiring high volume filling with fluid or fluidic suspension, such as in breast tissue and subcutaneous tumescence.

It should be appreciated that the invention can also have a fluid injection system including a series of valves, tubing, syringe and NaCL 0.9% IV fluid reservoie.

As shown in FIG. 3, the apparatus 1 may include a plastic sleeve 11, to protect the needles 2 when not in use.

To demonstrate the efficiency of the instant invention compared to the current standard, we performed flow velocity measurements in a single-blind study as shown in Table 1. The measurements for the current standard were taken using a BRAUN brand 21 gauge winged needle infusion set 7B3050. The instant invention incorporated four 21 gauge hypodermic needles.

TABLE 1
Flow velocity data of the instant invention and the current
standard reported as time to inject and the corresponding time savings
of using the instant invention rather than the current standard.
Multiple Injector Needle vs. Braun 21 g Injector Data (in seconds)

| | Multiple Injector | | |
|---|---|---|---|
| | Needle - Time to | Braun 21 g - Time to | |
| Simulation | Inject | Inject | Time Savings |
| 1 | 6.600 | 25.500 | 18.900 |
| 2 | 7.000 | 29.000 | 22.000 |
| 3 | 6.900 | 27.800 | 20.900 |
| 4 | 6.800 | 26.800 | 20.000 |
| 5 | 6.900 | 25.500 | 18.600 |
| 6 | 7.100 | 27.200 | 20.100 |
| 7 | 6.200 | 26.500 | 20.300 |
| 8 | 6.300 | 27.300 | 21.000 |
| 9 | 6.800 | 28.400 | 21.600 |
| 10 | 6.500 | 28.200 | 21.700 |
| 11 | 5.300 | 22.600 | 17.300 |
| 12 | 5.400 | 24.300 | 18.900 |
| 13 | 5.700 | 23.900 | 18.200 |
| 14 | 5.300 | 23.100 | 17.800 |
| 15 | 5.400 | 24.400 | 19.000 |
| 16 | 5.200 | 23.900 | 18.700 |
| 17 | 5.500 | 24.500 | 19.000 |
| 18 | 5.300 | 23.800 | 18.500 |
| 19 | 6.100 | 23.600 | 17.500 |
| 20 | 5.200 | 24.200 | 19.000 |
| Ave. | 6.075 | 25.525 | 19.450 |
| Std. Dev. | 0.709 | 1.963 | 1.419 |

As shown in Table 1, the evaluation conducted showed that the multiple injector needle average flow rate is 4.2-fold greater than the BRAUN 21 g winged needle infusion set 7B3050. The average time for injection for twenty (20) trials wherein the apparatuses were filled with 60 cc of fluid was 6.075 seconds for the multiple injector needle, while the BRAUN 21 g standard took 25.525 seconds. The standard deviation for each apparatus was 0.71 and 1.96 seconds, respectively. The two-tailed P value for this evaluation was less than 0.0001, which, considered by conventional criteria, is an extremely statistically significant difference. Also, the mean of the multiple injector needle minus the current standard equals 19.450 seconds with a 95% confidence interval for this difference from 18.507 to 20.393. it should be appreciated that the instant invention using four needles results in greater than fourfold increase in injection rate.

Based on the results of this evaluation, the greater than four times increased flow velocity of the instant invention results in the tedious and painful injection process being much more comfortable to perform. With less force required for injection, less body bruising from pressing against the ribs or abdomen, and less thumb injury will result. For instance, the basal joint of the thumb sees a thirteen (13) times increased load from that created at the site of compression. If you press the syringe with your thumb with twenty (20) pounds per square inch of pressure, your thumb basal joint sees two hundred and sixty (260) pounds per square inch, decreasing the risk of the development of arthritis due to conducting such an injection. Most importantly, faster injection corresponds to faster surgery time with the patient under anesthesia for less time. Accordingly, patients will have better recovery periods, lower chances for infection, and likely faster discharge from the hospital due to the time savings provided by the instant invention.

Further, the faster process associated with the multiple injector needle will likely save total procedure time resulting in cost savings. For instance, in a bilateral reconstruction procedure, the faster process will likely save upwards of ten (10) minutes with moderate intraoperative fill volumes. This cost savings is quantified via a 2005 peer-reviewed study with average cost of $62.00 per minute. This cost does not include anesthesia costs, nor the expense of consumables. Thus, actual savings are even higher.

Also, as shown in Table 2, we measured fluid released from the port during injection to determine leakage of the instant invention.

TABLE 2
Leakage data of the multiple injector needle after being
inserted and removed into the center of a port thirty (30) times
Leakage Data Log

| Day | Leak |
|---|---|
| 1-3 | No |
| 4-6 | No |
| 7-9 | No |
| 10-12 | No |
| 13-15 | No |
| 16-18 | No |
| 19-21 | No |
| 22-24 | No |
| 25-27 | No |
| 28-30 | No |
| 31-33 | No |
| 34-37 | No |
| 38-40 | No |
| 41-43 | No |
| 44-47 | No |
| 48-50 | No |
| 51-53 | No |
| 54-57 | No |
| 58-60 | No |
| 61-63 | No |
| 64-66 | No |
| 67-69 | No |
| 70-72 | No |
| 73-75 | No |
| 76-78 | No |
| 79-81 | No |
| 82-84 | No |
| 85-87 | No |
| 88-90 | No |
| 91-93 | N/A |

As shown in Table 2, no tissue expanders were found to be leaking following ninety (90) days of inspection. The multiple injector needle was used in three (3) different tissue expander samples (1 Mentor; 2 Allergan), and fluid was injected into the apparatus as well. The apparatus was turned upside down so that the port was at the lowest point and then wiped dry. Compression of the multiple injector needle with manual pressure was performed with the port at the lowest point in relationship to gravity. Visible and palpable testing was performed to look for any evidence of fluid released from the port. The lack of data leakage associated with the instant invention further indicates the reliability and efficiency of the design of the instant invention.

Although the invention has been described herein as being used with breast tissue expander, it is contemplated that it can be used in any tissue expander or in any other device with an injection port that can accommodate multiple needles.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an option (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

1. A fluid injection device for filling a tissue expander comprising:
a plurality of needles, each being in fluid communication with a respective needle base;
a hub having a first end portion and a second end portion, the plurality of needles projecting from the first end portion of the hub; and
a tubing extending from the hub and configured to attach the hub to a fluid source, wherein the plurality of needles comprises four or more needles axially aligned such that each of the needles can be inserted into an injection port of a tissue expander simultaneously, and wherein the needles are sized and spaced to allow an increased flow rate compared to a single needle without causing leakage from the injection port.

2. The device of claim 1, wherein the plurality needles comprises four needles.

3. The device of claim 1, wherein each needle comprises a 21 gauge needle.

4. The device of claim 1 or wherein the first end portion of the hub has a diameter of 3 cm or less.

5. The device of claim 1, further comprising a fluid source, wherein the fluid source comprises a syringe.

6. The device of claim 21, wherein the hub includes handles extending therefrom for enabling easier insertion of the needles into an injection port.

7. The device of claim 1, wherein each of the plurality of needles comprises an ultra-thin wall needle.

8. The device of claim 1, wherein each of the plurality of the needles comprises a longitudinal length from the hub to a tip of the needle, the longitudinal length of each of the plurality of needles being approximately equal.

9. The device of claim 1. wherein the plurality of needles are spaced no more than about 5 millimeters apart.

10. The device of claim 1, wherein the hub has a diameter in the range of about 0.25 cm to about 3 cm in diameter.

11. The device of claim 1. wherein the plurality of needles are hypodermic needles.
